(19) United States
(12) Patent Application Publication
(10) Pub. No.: US 2022/0108784 A1
(43) Pub. Date: Apr. 7, 2022

(54) SYSTEM AND METHOD FOR ASSISTING IN MENTAL HEALTH THERAPY

(71) Applicants: Gabriella MORALI, Cupertino, CA (US); Angelina Alex ROOPA, Cupertino, CA (US); Vaishnavi SURESH, Cupertino, CA (US)

(72) Inventors: Gabriella MORALI, Cupertino, CA (US); Angelina Alex ROOPA, Cupertino, CA (US); Vaishnavi SURESH, Cupertino, CA (US)

(21) Appl. No.: 17/493,523

(22) Filed: Oct. 4, 2021

Related U.S. Application Data
(60) Provisional application No. 63/087,349, filed on Oct. 5, 2020.

(51) Int. Cl. G16H 20/70; G16H 40/67; G16H 10/60; G16H 10/20; G16H 50/70; G06F 16/26; A61B 5/00; A61B 5/024; A61B 5/16

(52) U.S. Cl. CPC G16H 20/70; G16H 40/67; G16H 10/60; G16H 10/20; G16H 50/70; G16H 15/00; A61B 5/0022; A61B 5/02405; A61B 5/165; A61B 5/7275; A61B 5/7278; G06F 16/26

(57) ABSTRACT

The present invention relates to a system (100) and a method for assisting in mental health therapy. The present invention includes a cloud server computer (102), a patient device (110), and a therapist device (108). The cloud server computer (102) includes a database unit (104) and a system processing unit (106). The patient device (110) displays multiple daily questionnaires regarding CBT and exposure therapy. The patient device (110) collects and communicates this data of the patient to be stored in the database unit and analyzed to create insights on the patient's mental health (104). At least one therapist device (108) receives data of the patient from at least one database unit (104). The system processing unit (106) generates a patient mental profile log and alert system. Further, the therapist device (108) receives a patient mental profile log in order to modify in person sessions.

### FIELD OF INVENTION

The present invention relates to a system and method for assisting in mental health therapy. More specifically, the present invention relates to cognitive behavioral therapy (CBT) logs, which allows the patient to log information geared towards enhancing their CBT journey.

Human beings live through a lot of different types of experiences during their lifetime. These experiences contain various people, places, and environments as part of them. Experiences encountered during a person's lifetime can contribute hugely to one's personal development and mental health.

They can also contribute towards maintaining a healthy mental state, as positive memories from the past can help people get inspired and motivated. Mental health refers to cognitive, behavioral, and emotional well-being. It is all about how people think, feel, and behave.

Looking after mental health can preserve a person's ability to enjoy life. Doing this involves reaching a balance between life activities, responsibilities, and efforts to achieve psychological resilience. Conditions such as stress, depression, and anxiety can all affect mental health and disrupt a person's routine.

People often take therapy sessions to improve their mental health, but it lacks an effective way to stay connected with the therapist when a person's mental health deteriorates. As technologies have improved, people have developed methods to connect the patient to the therapist via the internet. These are much more effective and are richer with information than weekly meetings with one's therapist, but they still lack an effective way to log day to day information about a person's mental health.

They are not able to rate within a specific range what emotions they faced. They also cannot record other details associated with a person's experiences such as his state of mind and feelings. Apart from external experiences and interactions, there are additional things associated with a person's life such as thoughts, ideas, and feelings. Though traditional journaling apps have methods of recording this, there is no system in place to analyze and quickly send the therapist meaningful insights into the patient's mental state based on the data collected. In addition, these applications do not provide a method of combining CBT and exposure therapy techniques in its features, which makes these applications ineffective for individuals with comorbid OCD or Anxiety.

Patent Application US2020082927A1 discloses a technological platform for delivering supervised and unsupervised digital behavior therapies to patients suffering from any one of a variety of maladies, including but not limited to stress, anxiety, addiction, and depression. The platform includes a server computer (110), a database (120), a mobile patient device (150), and a therapist device (160). The mobile patient device comprises a processor, memory, an audio system, and a mobile app (the “With Hope” app) that configures the mobile patient device to deliver selected behavior therapies to a patient, including a selected binaural beat designed to induce a selected brain wave in the patient in combination with a selected set of subliminal affirmations. Alternatively, the mobile patient device could be configured to deliver a selected binaural beat designed to induce a selected brain wave in the patient in combination with exercises in meditation.

Patent Application WO2016071660A1 discloses apparatus for use in a computer-based system for providing psychological therapy, the apparatus comprising: an access system configured to control access to features and data by users of remote devices, wherein the users comprise patients, therapists and supervisors; and a therapy system configured to enable text-based instant messages to be sent between patients and therapists; wherein the access system is configured to allow patients to retrieve messages sent and received by the patient, therapists to retrieve messages sent and received by the therapist, and supervisors to retrieve messages sent and received by particular patients and/or therapists.

The existing inventions are not able to overcome the problem associated with the method to find a patient's trigger activity and work on it. The existing inventions are complex and are not cost-effective. Thus there is a need for the present invention to overcome the above mentioned problems. In addition, these inventions do not record and analyze CBT data for the use of the therapist. This means that the time in between sessions is not often accounted for, and none of the aforementioned inventions solve this issue.

### SUMMARY OF THE INVENTION

The present invention relates to a system and method for assisting in mental health therapy. The present invention includes a cloud server computer, a patient device, and a therapist device. The cloud server computer includes a database unit and a system processing unit. The database unit stores computer-readable instructions, data of a patient, and data of the therapist. The system processing unit executes computer-readable instructions that are stored in the database unit.

The patient device stores computer-readable instructions that are executed by the patient device to communicate with the system processing unit of the cloud server computer. The therapist device stores computer-readable instructions that are executed by the therapist device to communicate with the system processing unit of the cloud server computer. The therapist device has access to the data provided by the patient.

Herein, the patient device communicates the data of the patient to be stored in the database unit of the cloud server computer. The therapist administers a patient account, which allows the patient to log into the patient device to communicate with the system processing unit of the cloud server computer. The patient device generates exposure logs, CBT logs, journaling features, and emergency logging and alert systems. In embodiment, the patient is able to sync any heart rate monitor to the system processing unit of the cloud server computer. This data is then stored in a database unit and is analyzed to provide certain insights about the patient's mental state. The heart rate data is compared to the patient's input to predict when a patient is feeling anxious or overwhelmed based on spikes in their heart rate. Both the raw data and the insights are sent to the therapist device. The therapist device receives data of the patient's input in between sessions from the database unit and the therapist gains insight on the patient's mental state. The system processing unit generates a patient mental profile log, and alert system based on the mental health diagnosis.

The main advantage of the present invention is that the present invention assists in mental health therapy.

Another advantage of the present invention is that the present invention provides the therapist's visibility to the patient's mental state in between sessions.

Yet another advantage of the present invention is that the present invention is an easy and cost-effective device.

Yet another advantage of the present invention is that the present invention uses questionnaires to guide the patient through the CBT process.

Yet another advantage of the present invention is that the present invention collects heart rate data to measure a patient's anxiety levels.

Yet another advantage of the present invention is that the present invention works on the concept of a patient's motivation.

Further objectives, advantages, and features of the present invention will become apparent from the detailed description provided hereinbelow, in which various embodiments of the disclosed invention are illustrated by way of example.

### BRIEF DESCRIPTION OF THE DRAWINGS

The accompanying drawings are incorporated in and constitute a part of this specification to provide a further understanding of the invention. The drawings illustrate one embodiment of the invention and together with the description, serve to explain the principles of the invention.

FIG. 1 illustrates the system of the present invention.

### DETAILED DESCRIPTION OF THE INVENTION

### Definitions

The terms “a” or “an”, as used herein, are defined as one or as more than one. The term “plurality”, as used herein, is defined as two as or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended. The term “comprising” is used interchangeably used by the terms “having” or “containing”.

Reference throughout this document to “one embodiment”, “certain embodiments”, “an embodiment”, “another embodiment”, and “yet another embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics are combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps, or acts are in some way inherently mutually exclusive.

As used herein, the term “one or more” generally refers to, but not limited to, singular as well as the plural form of the term.

The drawings featured in the figures are to illustrate certain convenient embodiments of the present invention and are not to be considered as a limitation to that. The term “means” preceding a present participle of operation indicates the desired function for which there is one or more embodiments, i.e., one or more methods, devices, or apparatuses for achieving the desired function and that one skilled in the art could select from these or their equivalent because of the disclosure herein and use of the term “means” is not intended to be limiting.

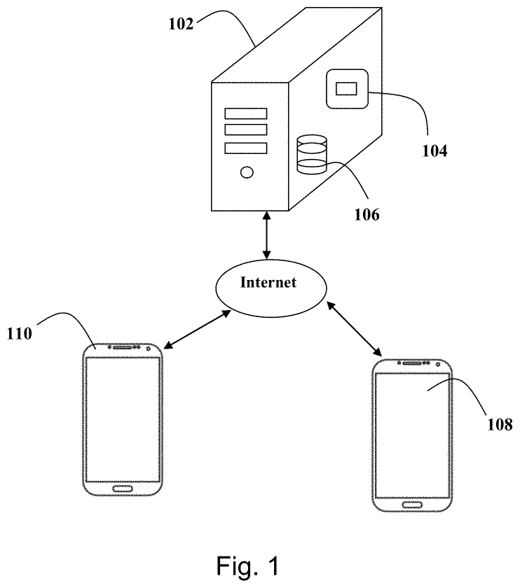

FIG. 1 illustrates a system (100) for assisting in mental health therapy. The system (100) includes a cloud server computer (102), a patient device (110), and a therapist device (108). The cloud server computer (102) includes a database unit (104), and a system processing unit (106). The database unit (104) stores computer-readable instructions, data of the patient, and data of the therapist. The system processing unit (106) executes computer-readable instructions that are stored in the database unit (104). The patient device (110) stores computer-readable instructions that are executed by the patient device (110) to communicate with the system processing unit (106) of the cloud server computer (102). The therapist device (108) stores computer-readable instructions that are executed by the therapist device (108) to communicate with the system processing unit (106) of the cloud server computer (102). The therapist device (108) having access to the data provided by the patient.

The present invention relates to a system and method for assisting in mental health therapy. The present invention includes a cloud server computer, a patient device, and a therapist device. The cloud server computer includes a database unit and a system processing unit. The database unit stores computer-readable instructions, data of the patient, and data of the therapist. The system processing unit executes computer-readable instructions that are stored in the database unit. The patient device stores computer-readable instructions that are executed by the patient device to communicate with the system processing unit of the cloud server computer. The therapist device stores computer-readable instructions that are executed by the therapist device to communicate with the system processing unit of the cloud server computer. The therapist device has access to the data provided by the patient.

Herein, the patient device communicates the data of the patient to be stored in the database unit of the cloud server computer. The therapist administers a patient account, which allows the patient to log into the patient device to communicate with the system processing unit of the cloud server computer. The patient device generates exposure logs, CBT logs, journaling features, and emergency logging and alert systems. In embodiment, the patient is able to sync any heart rate monitor to the system processing unit of the cloud server computer. This data is then stored in a database unit and is analyzed to provide certain insights about the patient's mental state. The heart rate data is compared to the patient's input to predict when a patient is feeling anxious or overwhelmed based on spikes in their heart rate. Both the raw data and the insights are sent to the therapist device. The therapist device receives data of the patient's input in between sessions from the database unit and the therapist gains insight on the patient's mental state. The system processing unit generates a patient mental profile log, and alert system based on the mental health diagnosis.

In an embodiment, the cloud server computer including, but not limited to, a desktop computer, a server, a mainframe computer. In an embodiment, the patient device and the therapist device are including, but not limited to, a tablet, a smartphone, and personal computer.

In an embodiment, the present relates to a method for assisting in mental health therapy, the method comprising:

A method of therapist profile generation, the method having;

a therapist creates a digital account on a cloud server computer with the help of a therapist device;

the therapist device communicates data of the therapist to be stored in a database unit of the cloud server computer; and

the therapist administers a code on the cloud server computer with the help of the therapist device.

A method of a patient profile generation, the method having

the therapist administers the code to a patient device with the help of the therapist device; and

by using administer code a patient creates a digital account on the cloud server computer with the help of the patient device.

A method for assisting in mental health therapy, the method having

after the creation of the digital account, the patient device displays multiple questionnaire that needs to be filled to generate the patient's mental profile log;

one such questionnaire is a CBT log, in which a patient is asked daily about what meal they are consuming, how they feel, their cognitive distortions, what their thoughts are, and what their counter thoughts are;

another such questionnaire is an exposure log, in which a patient is asked daily about what triggering activity they are participating in and how they felt;

another such questionnaire is the emergency log, in which a patient logs whether they purged, used laxatives, cut, and any other harmful activity;

data is also collected through the use of journal features, in which a patient can record how they feel for a therapist to access;

with the help of the patient device, the patient fills up the answers to the questionnaire and communicates data to be stored in the database unit of the cloud server computer;

the system processing unit executes computer-readable instructions and analyzes the patient data to create insights on the patient's mental state;

the patient data and insights are sent to one or more therapist devices from the one or more database units;

based on the patient input, the system processing unit executes computer-readable instructions to generate the patient's mental profile log that includes average mood rating, run streaks, trigger activities, common cognitive distortions, and an editable graph on mood profile and also generates a page for contact and journal;

with the help of the therapist device, the therapist has access to the patient's mental profile log displaying patients' average mood rating, run streaks, age, name, diagnosis, trigger activities, common cognitive distortions, and an editable graph on mood profile and patients' contact page, journal;

with the help of the patient device, the patient has access to the patient's mental profile log displaying patients' average mood rating, run streaks, age, name, diagnosis, trigger activities, common cognitive distortions, and a graph on the mood profile, and their journals;

Herein, the therapist monitor progress in the patient mental health over time, by repetitively getting the answer to the questionnaire on a regular interval;

Herein, based on the patient's mental profile log, the therapist improves the therapy for the patients.

A method for emergency logging and alert system, the method having

the patient device is provided with an emergency logging feature for the patient. If the patient has done any act that could potentially hurt themselves, the patient is able to log such emergency using emergency logging feature;

the system processing unit executes computer-readable instructions to generate an alert system;

if a patient has consistently been feeling down based on the answers of the questionnaire and frequency of emergency logging, the therapist and as well as the patient receive an alert message on the therapist device and the patient device.

Herein, in the therapist device, there is a feature to call the patient in case the alert message is received. Also, the patient device is provided with a hotline page having contact details of the therapist and another emergency service.

Herein, the patient device is able to show the patient their run streak, that is how long the patient has consistently been logging their behavior on the database unit;

Herein, the therapist device has a feature to write notes related to the patient, which is published similarly to the journal on the patient's mental profile log;

In the preferred embodiment, the patient's mental profile log is based on the cognitive-behavioral therapy (CBT) logs, exposure logs, and emergency logs.

In a preferred embodiment, the patient device generates the exposure logs for the patients to fill out to record what triggering activity the patient participated in and their rating of the activity. The exposure logs help the patient to find the activities with the lowest ratings consistently.

In the preferred embodiment, a graph on the mood profile is being displayed on the patient device to see the progress of the therapy.

In a preferred embodiment, the patient is able to sync any heart rate monitor to the system processing unit of the cloud server computer. This data is then stored in a database unit and is analyzed to provide certain insights about the patient's mental state. The heart rate data is compared to the patient's input to predict when a patient is feeling anxious or overwhelmed based on spikes in their heart rate. Both the raw data and the insights are sent to the therapist device and patient device

In a preferred embodiment, there are two major alerts in the alert system, that are the yellow alert and red alert, the red alert shows up if the patient has been excessively logging emergencies, then the therapist and as well as the patient receive an alert message on the therapist device and the patient device.

Herein, the yellow alert is generated when the patient end user has consistently been feeling down based on the answers of the various questionnaires.

In an embodiment, the present invention relates to a system and method for assisting in mental health therapy. The present invention includes one or more cloud server computers, one or more patient devices, and one or more therapist devices. One or more cloud server computers include one or more database units and a system processing unit. One or more database units store computer-readable instructions, data of the patient, and data of the therapist. The system processing unit executes computer-readable instructions that are stored in one or more database units. One or more patient devices store computer-readable instructions that are executed by one or more patient devices to communicate with the system processing unit of one or more cloud server computers. One or more therapist devices store computer-readable instructions that are executed by one or more therapist devices to communicate with the system processing unit of the at least cloud server computers. One or more therapist devices are having access to the data provided by the patient.

Herein, the one or more patient devices communicate the data of the patient to be stored in the one or more database units of the cloud server computer. The therapist administers a patient account, which allows the patient to log into the one or more patient devices to communicate with the system processing unit of the one or more cloud server computers. The one or more patient devices generate exposure logs, CBT logs, journaling features, and emergency logging and alert systems. In embodiment, the patient is able to sync any heart rate monitor to the system processing unit of the one or more cloud server computers. This data is then stored in the one or more database units and is analyzed to provide certain insights about the patient's mental state. The heart rate data is compared to the patient's input to predict when a patient is feeling anxious or overwhelmed based on spikes in their heart rate. Both the raw data and the insights are sent to the one or more therapist devices. The one or more therapist devices receive data of the patient's input in between sessions from the database unit and the therapist gains insight on the patient's mental state. The system processing unit generates a patient mental profile log, and alert system based on the mental health diagnosis.

In an embodiment, one or more cloud server computers including, but not limited to a desktop computer, a server, a mainframe computer. In an embodiment, one or more patient devices and one or more therapist devices are including, but not limited to, a tablet, a smartphone, and a touch-based personal computer.

In an embodiment, the present relates to a method for assisting in mental health therapy, the method comprising:

A method of therapist profile generation, the method having

a therapist creates a digital account on one or more cloud server computers with the help of one or more therapist devices;

one or more therapist devices communicate data of the therapist to be stored in one or more database units of one or more cloud server computers;

and

the therapist administers a code on one or more cloud server computers with the help of one or more therapist devices.

A method of a patient profile generation, the method having

the therapist administers a code to one or more patient devices with the help of one or more therapist devices; and

by using administer code a patient creates a digital account on one or more cloud server computers with the help of one or more patient devices.

A method for assisting in mental health therapy, the method having;

after the creation of the digital account, one or more patient devices displays a questionnaires that needs to be filled to generate the patient's mental profile log;

one such questionnaire is a CBT log, in which a patient is asked daily about what meal they are consuming, how they feel, their cognitive distortions, what their thoughts are, and what their counter thoughts are;

another such questionnaire is an exposure Log, in which a patient is asked daily about what triggering activity they are participating in and how they felt;

another such questionnaire is the emergency log, in which a patient logs whether they purged, used laxatives, cut, and any other harmful activity;

Data is also collected through the use of journal features, in which a patient can record how they feel for a therapist to access;

with the help of one or more patient devices, the patients fill up the answers to the questionnaire and communicate data to be stored in one or more database units of one or more cloud server computers;

the system processing unit executes computer-readable instructions and analyzes the patient data to create insights on the patient's mental state;

the patient data and insights are sent to one or more therapist devices from the one or more database units;

based on the patient's data, the system processing unit executes computer-readable instructions to generate the patient's mental profile log that includes average mood rating, run streaks, trigger activities, common cognitive distortions, and an editable graph on mood profile and also generate a page for contact and journal;

with the help of one or more therapist devices, the therapist has access to the patient's mental profile log displaying patients' average mood rating, run streaks, age, name, diagnosis, trigger activities, common cognitive distortions, and an editable graph on mood profile and patients' contact page, journal;

with the help of one or more patient devices, the patient has access to the patient's mental profile log displaying patients' average mood rating, run streaks, age, name, diagnosis, trigger activities, common cognitive distortions, a graph on mood profile, a patients' contact page, and a journal.

Herein, the therapist monitors progress in a patient's mental health over time, by repetitively getting the answer to the questionnaire on a regular interval and updating the patient's mental profile log.

Herein, based on the patient's mental profile log, the therapist improves the therapy for the patients.

A method for emergency logging and alert system, the method having

one or more patient devices are provided with an emergency logging feature for the patient, if patient has done any act that could potentially hurt themselves the patient is able to log such emergency using emergency logging feature;

the system processing unit executes computer-readable instructions to generate an alert system;

if a patient has consistently been feeling down that is being analyzed through the answer of the questionnaire and frequency of emergency logging, the therapist and as well as the patient receive an alert message on one or more therapist devices and one or more patient devices,

Herein, in one or more therapist devices there is a feature to call the patient in case the alert message is received. Also, one or more patient devices are provided with a hotline page having contact details of the therapist and other emergency services.

Herein, one or more patient devices are able to show the patient their run streak, which is how long the patient has consistently been logging their behavior on one or more database units.

Herein, one or more therapist devices have a feature to write notes related to the patient, which is published similarly to the journal on the patient's mental profile log.

In the preferred embodiment, the patient's mental profile log is based on the cognitive-behavioral therapy (CBT) logs, exposure logs, and emergency logs.

In the preferred embodiment, patients, with the help of one or more patients devices, generate the exposure logs for the patients to fill out log to record what triggering activity the patient participated in and their rating of the activity. The exposure logs help the patient to find the activities with the lowest ratings consistently.

In the preferred embodiment, a graph on mood profile is being displayed on one or more patient devices to see the progress of the therapy.

In the preferred embodiment, the patient is able to sync any heart rate monitor to the system processing unit of the cloud server computer. This data is then stored in a database unit and is analyzed to provide certain insights about the patient's mental state. The heart rate data is compared to the patient's input to predict when a patient is feeling anxious or overwhelmed based on spikes in their heart rate. Both the raw data and the insights are sent to the therapist device.

In the preferred embodiment, two major alerts in the alert system, that are the yellow alert and red alert, the red alert shows up if the patient has been excessively logging emergencies, then the therapist and as well as the patient receive an alert message on one or more therapist devices and one or more patient devices.

Herein, the yellow alert is generated when the patient end user has consistently been feeling down based on the answers of the various questionnaires.

Further objectives, advantages, and features of the present invention will become apparent from the detailed description provided herein, in which various embodiments of the disclosed present invention are illustrated by way of example and appropriate reference to accompanying drawings. Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings.

I/We claim:

1. A system (100) and method for assisting in mental health therapy, the system (100) comprising:
at least one cloud server computer (102), the at least one cloud server computer (102) having
an at least one database unit (104), the at least one database unit (104) stores computer-readable instructions, data of patient and data of therapist, and
a system processing unit (106), the system processing unit (106) executes computer-readable instructions that are stored the at least one database unit (104);
an at least one patient device (110), the at least one patient device (110) stores computer-readable instructions that are executed by the at least one patient device (110) to communicate with the system processing unit (106) of the at least one cloud server computer (102); and
an at least one therapist device (108), the at least one therapist device (108) stores computer-readable instructions that are executed by the at least one therapist device (108) to communicates with the system processing unit (106) of the at least one cloud server computer (102), the at least one therapist device (108) having access to the data provided by the patient;
wherein, the therapist administers a patient account, which allows the patient to log in to the at least one patient device (110) to communicate with the system processing unit (106) of the at least one cloud server computer (102). The patient's device generates exposure logs, CBT logs, journaling features, and emergency logging and alert systems.
wherein, the at least one patient device (110) communicates the data of the patient to be stored in the at least one database unit (104) of the at least one cloud server computer (102),
wherein, at least one therapist device (108) receives data and insights of the patient's mental state from the at least one database unit (104),
wherein, the system processing unit (106) generates a patient mental profile log, alert system based on the mental health data.

2. The system (100) as claimed in claim 1, wherein at least one cloud server computer (102) is selected from a desktop computer, a server, a mainframe computer.

3. The system (100) as claimed in claim 1, wherein the at least one patient device (110) and the at least one therapist device (108) are selected from a tablet, a smartphone, and a personal computer.

4. A method designed to assist mental health therapy, the method comprising:
a method of therapist profile generation, the method having
a therapist creates a digital account on an at least one cloud server computer (102) with the help of an at least one therapist device (108),
the at least one therapist device (108) communicates data of the therapist to be stored in an at least one database unit (104) of an at least one cloud server computer (102), and
the therapist administers a code on the at least one cloud server computer (102) with the help of the at least one therapist device (108);
a method of a patient profile generation, the method having
the therapist administers the code to an at least one patient device (110) with the help of the at least one therapist device (108), and
by using administer code a patient creates a digital account on an at least one cloud server computer (102) with the help of the at least one patient device (110);
a method for assistance in mental health therapy, the method having
after the creation of the digital account, the at least one patient device (110) displays multiple questionnaires that need to be filled to generate the patient's mental profile log,
one such questionnaire is a CBT log, in which a patient is asked daily about what meal they are consuming, how they feel, their cognitive distortions, what their thoughts are, and what their counter thoughts are, another such questionnaire is an exposure log, in which a patient is asked daily about what triggering activity they are participating in and how they felt,
another such questionnaire is the emergency log, in which a patient logs whether they purged, used laxatives, cut, and any other harmful activity,
Data is also collected through the use of journal features, in which a patient can record how they feel for a therapist to access,
with the help of the at least one patient device (110), the patient fills up the answers to the questionnaires and communicates data to be stored in the at least one database unit (104) of the at least one cloud server computer (102),
the system processing unit (106) executes computer-readable instructions send, answers to the questionnaire provided by the patient, to the at least one therapist device (108) from the at least one database unit (104),
based on the mental health data of patient based on the questionnaires, the system processing unit (106) executes computer-readable instructions to generate the patient's mental profile log that includes average mood rating, run streaks trigger activities, common cognitive distortions, and an editable graph on mood profile and also generate a page for contact and journal,
with the help of the at least one therapist device (108), the therapist has access to the patient's mental profile log displaying patients' average mood rating, run streaks, age, name, diagnosis, trigger activities, common cognitive distortions, and an editable graph on mood profile and patients' contact page, journal, and
with the help of the at least one patient device (110), the patient has access to the patient's mental profile log displaying patients' average mood rating, run streaks, age, name, diagnosis, trigger activities, common cognitive distortions, and a graph on mood profile and patients' contact page, journal;
wherein, the therapist monitors progress in patients' mental health over time, by repetitively getting the answer to the questionnaire on a regular interval and updating the patient's mental profile log,
wherein, based on the patient's mental profile log, the therapist improves the therapy for the patients;
a method for emergency logging and alert system, the method having
the at least one patient device (110) is provided with an emergency logging feature for the patient, if the patient has done any act that could potentially hurt themselves the patient is able to log such emergency using emergency logging feature;
the system processing unit (106) executes computer-readable instructions to generate an alert system, wherein, if a patient has consistently been feeling down based on the answers of the questionnaire and frequency of emergency logging, the therapist and as well as the patient receive an alert message on the at least one therapist device (108) and the at least one patient device (110), wherein, in the at least one therapist device (108) there is a feature to call the patient in case the alert message is received, and also the at least one patient device (110) is provided with hotline page having contact details of the therapist and other emergency services;
wherein, the at least one patient device (110) is able to show the patient their run streak, that is how long the patient has consistently been logging their behavior on the at least one database unit (104),
wherein, the at least one therapist device (108) has a feature to write notes related to the patient, which is published similarly to the journal on the patient's mental profile log.

5. The method as claimed in 4, the patient's mental profile log is based on the cognitive-behavioral therapy (CBT) logs, exposure logs, and emergency log.6. The method as claimed in 4, at least one patient device (110), generates the exposure logs for the patients to fill out log to record what triggering the patient participated in and their rating of the activity, wherein, exposure logs help the patient to find the activities with the lowest ratings consistently.

6. The method as claimed in claim 4, wherein, a graph on mood profile is being displayed on at least one patient device (110) to see the progress of the therapy.

7. The method as claimed in claim 4, wherein, the patients, with the help of at least one patient device (110), the patient is able to sync any heart rate monitor to the system processing (106) unit of the at least one cloud server computer (102), this data is then stored in the at least one database unit (104) and is analyzed to provide certain insights about the patient's mental state, the heart rate data is compared to the patient's input to predict when a patient is feeling anxious and overwhelmed based on spikes in their heart rate, both the raw data and the insights is sent to the at least one therapist device (108).

8. The method as claimed in claim 4, wherein, are two major alerts in the alert system, that are the yellow alert and red alert, wherein, the red alert shows up if the patient has been excessively logging emergencies, then the therapist and as well as the patient receive an alert message on the at least one therapist device (108) and the at least one patient device (110) wherein, the yellow alert is generated when the patient end user has consistently been feeling down based on the answers of the various questionnaires.
